(19) United States
(12) Patent Application Publication
(10) Pub. No.: US 2012/0203335 A1
(43) Pub. Date: Aug. 9, 2012

(54) COLLAPSIBLE DOCKING STATION

(76) Inventors: Ivan Vesely, Larkspur, CO (US); Christopher Michael Sprague, Broomfield, CO (US); Dean Carpenter, Boulder, CO (US)

(21) Appl. No.: 13/500,359

(22) PCT Filed: Oct. 13, 2010
(86) PCT No.: PCT/US2010/052451

§ 371 Date: Apr. 5, 2012

Related U.S. Application Data
(60) Provisional application No. 61/251,387, filed on Oct. 14, 2009.

(51) Int. Cl. A61F 2/24

(52) U.S. Cl. 623/2.18

(57) ABSTRACT

A collapsible docking station for a valve assembly that includes the collapsible docking station and an exchangeable valve member detachably coupled thereto. The collapsible docking station includes a band that is moveable between a collapsed position where the band forms a wound coil, and a fully expanded position where the band forms a circular ring.

### RELATED APPLICATION

This application claims the benefit of U.S. Provisional Application No. 61/251,387, filed Oct. 14, 2009, and is fully incorporated herein by reference.

### FIELD OF THE INVENTION

The present invention relates generally to the field of cardiovascular valves, and more particularly to a collapsible docking station for a valve assembly comprised of the collapsible docking station and an exchangeable valve member detachably coupled thereto.

### BACKGROUND OF THE INVENTION

Evidence that transcatheter valves are likely to have serious durability issues due to geometry first came to light in a paper by Rachid Zegdi of Paris, France, published in the American Journal of Cardiology in 2008 (J Am Coll Cardiol 2008; 51:579-584). After obtaining informed consent from patients undergoing conventional, open-heart surgery to implant a prosthetic valve, researchers inserted empty Nitinol transcatheter valve stents into the orifice of native calcified, stenotic aortic valves and photographed their expanded shape. Because of the presence of the calcified leaflets, noncircular deployment of the stent occurred in 86% of all deployments into bicuspid valves and in 32% of all hi-leaflet valves. However, even when deployed in tri-leaflet valves to a circular shape, the range of deployed diameters was 17-20 mm for a valve designed for a nominal deployed geometry of 20 mm. A visible gap between the expanded stent and the adjacent tissue was noted in 49% of all cases and the protrusion of calcific nodules through the Nitinol cage was found in the majority of cases. To visualize the impact of the non-circular and under-deployed geometry on the shape of the leaflets, the researchers then inserted transcatheter valves into plastic forms that mimicked the geometries observed during the open-heart procedures. A wide range of leaflet distortions were observed.

Profound distortions in leaflet geometry lead to concerns as to whether grossly distorted valves will have durability beyond just a few years. The reasons for non-circular deployment are understood as follows. The final size and shape of the deployed valve is determined by the elastic balance between the expanding Nitinol stent and the asymmetric resistance of the calcified leaflets of the native aortic valve. The final shape (e.g., triangular, circular or elliptical) and the final diameter of the self-expanding valve depends on how the native valve is diseased, where the calcific nodules are, and how asymmetrically positioned they may be. Existing self-expanding valves (e.g., CoreValve and Ventor from Medtronic, Inc.) are likely to be most vulnerable to such geometrical abnormalities since their shape is determined primarily by the elastic balance between the expansion of the Nitinol cage and the recoil of the stenotic valve. It has been observed with conventional, current generation, state-of-the-art pericardial valves (e.g., Perimount from Edwards Lifesciences LLC) that even a 1 mm departure from absolute geometrical symmetry leads to rapid leaflet degeneration and early valve failure.

Unfortunately, the geometry of the valve after deployment in a patient cannot be fully visualized with current imaging technologies. Transesophageal echocardiography does not have resolution sufficient to show observed pinwheeling and leaflet distortion. Transcatheter valves typically function well for the first several years, but with grossly misshapen leaflets, experience dictates that they cannot function well beyond that amount of time.

Transcatheter valves are an important life-extending option for the aged, inoperable patient. Such valves will not become an option for the otherwise healthy, operable patient who expects 15-year longevity from a bioprosthetic valve. In order to assure such longevity, there is a need for a new valve design with precisely-controlled leaflet geometry.

The present invention addresses these and other drawbacks of existing valve designs by providing a collapsible docking station for a valve assembly comprised of the collapsible docking station and an exchangeable valve member that is detachably coupled thereto.

### SUMMARY OF THE INVENTION

In accordance with one aspect of the present invention, there is provided a collapsible docking station engageable with a detachable valve member having valve leaflets, said collapsible docking station including: a band moveable between a collapsed position and an expanded position; and a plurality of posts adapted to engage with the detachable valve member.

An advantage of the present invention is the provision of a collapsible docking station that allows a detachable valve member to have a substantially circular, rigid configuration when coupled to the docking station, as compared to existing transapically insertable valves.

Another advantage of the present invention is the provision of a docking station for receiving a detachable valve member, wherein the docking station includes a frame element having an adjustable diameter such that the docking station is moveable between a collapsed position and an expanded position.

These and other advantages will become apparent from the following description taken together with the accompanying drawings and the appended claims.

### BRIEF DESCRIPTION OF THE DRAWINGS

The invention may take physical form in certain parts and arrangement of parts, an embodiment of which will be described in detail in the specification and illustrated in the accompanying drawings which form a part hereof, and wherein:

FIG. 1 is an exploded view of a valve assembly comprised of an exchangeable valve member and a collapsible docking station according to an embodiment of the present invention;

FIG. 2 is a perspective view of the valve assembly of FIG. 1 with the exchangeable valve member installed on the docking station;

FIG. 3 is a perspective view of the docking station in a collapsed position, and engaged by an installation tool;

FIG. 4 is a perspective view of the docking station in an expanded position, and engaged by the installation tool;

FIG. 5 is a perspective view of the docking station in an expanded position, and disengaged from the installation tool;

FIG. 6 is a cross-sectional top plan view of the docking station in a fully collapsed position;

FIG. 7 is a cross-sectional top plan view of the docking station in an expanded position;

FIG. 8 is a perspective view of a fully expanded metal band of the docking station;

FIG. 9 is a perspective view of a post of the docking station;

FIG. 10 is a cross-sectional top plan view of a partially collapsed metal band and a single fixed post;

FIG. 11 is a cross-sectional top plan view of a partially collapsed metal band and a pair of floating posts;

FIG. 12 is a perspective view of a fully expanded convex metal band;

FIG. 13 is a perspective view of a fully expanded concave metal band;

FIG. 14 is a perspective view of a collapsible docking station according to an alternative embodiment of the present invention; and

FIG. 15 is a cross-sectional view of a section of the docking station taken along lines 15-15 of FIG. 14.

### DETAILED DESCRIPTION OF THE INVENTION

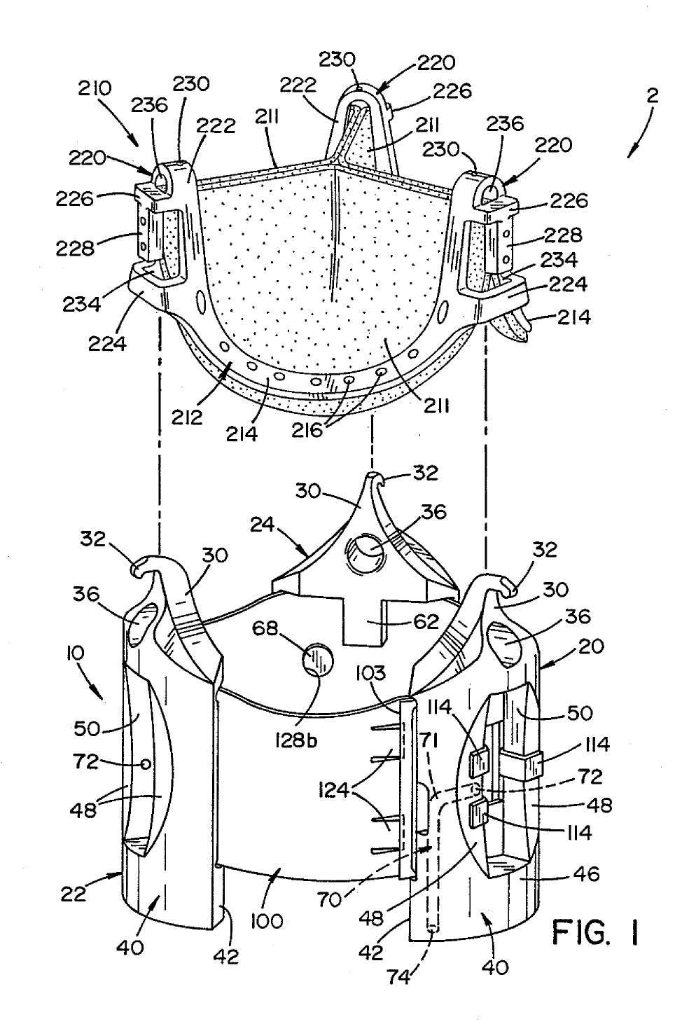

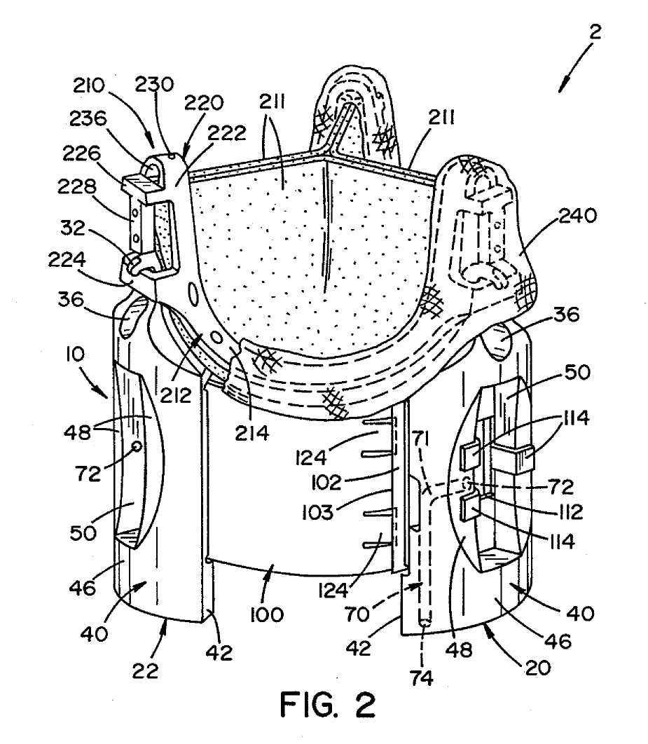

Referring now to the drawings wherein the showings are for the purposes of illustrating an embodiment of the invention only and not for the purposes of limiting same, FIG. 1 shows an exploded view of a cardiovascular valve assembly 2 that includes a collapsible base member or docking station 10 according to an embodiment of the present invention, and an exchangeable valve member 210 that is adapted to be detachably coupled to docking station 10. FIG. 2 shows valve member 210 coupled to docking station 10.

It should be appreciated that valve member 210, shown in FIGS. 1 and 2, is intended as an example of a valve member adapted for use in connection with the docking station of the present invention, and is not intended to limit the scope of the present invention. Accordingly, the docking station of the present invention is also intended for use in connection with valve members having alternative designs. Furthermore, while the illustrated embodiment shows valve member 210 as a bioprosthetic valve, it is contemplated that the valve member used in connection with the present invention may also take the form of a mechanical valve.

Docking station 10 is generally comprised of a plurality of posts, i.e., fixed post 20 and floating posts 22 and 24, and a collapsible metal band 100. Each post 20, 22 and 24 is comprised of a mounting or engagement portion 30 and a main body 40. It should be appreciated that the number of fixed and floating posts may vary without departing from the spirit and scope of the present invention. In the illustrated embodiment, engagement portion 30 includes a protuberance 32 in the form of a hook or tab for coupling valve member 210 to docking station 10. Main body 40 is adapted to attach the post to band 100, as will be described below. A sewing cuff or ring (not shown) made of Dacron®, or other medical grade fabric, may be sewn to the outer surface of docking station 10. The sewing cuff or ring permanently attaches docking station 10 to the tissue of the heart. Docking station 10 will be described in further detail below.

Valve member 210 is generally comprised of a frame 212 and a plurality of valve leaflets 211 (i.e., a leaflet set) supported by frame 212. Frame 212 includes a plurality of ribbon sections 214 and coupling elements 220. Coupling elements 220 function as stent posts of valve member 210, and allow valve member 210 to be coupled and uncoupled from docking station 10, as will be described below.

In the illustrated embodiment, each coupling element 220 is comprised of a generally U-shaped portion 222 having lower and upper crossbars 224, 226 extending across U-shaped portion 222. Upper crossbar 226 is T-shaped and includes a downward extending finger 228. Finger 228 and lower crossbar 224 define a lower slot 234. Upper crossbar 226 and the top section of U-shaped portion 222 define an upper slot 236. In one embodiment of valve member 210, each coupling element 220 includes an opening 230 in generally U-shaped portion 222.

Each ribbon section 214 has a generally arcuate shape, and extends between coupling elements 220. Ribbon sections 214 are dimensioned to form a seal with docking station 10 when valve member 210 is coupled thereto, as shown in FIG. 2. This seal prevents blood leakage between valve member 210 and docking station 10.

Frame 212 is preferably made of a flexible material having suitable elasticity to allow frame 212 to collapse into a tight bundle for convenient removal and exchange of valve member 210 through small incisions or a trocar, and to facilitate the engagement and disengagement of coupling elements 220 and engagement portions 30. Frame 212 is preferably made of a medical grade polymer material, such as poly-ether-ether-ketone (PEEK), polyurethane or polycarbonate. However, it is also contemplated that frame 212 may alternatively be formed of a metal, including, but not limited to, Elgiloy, nitinol, stainless steel, platinum, gold, titanium, other biocompatible metals, and combinations thereof. A fabric cover 240 made of a medical grade cloth may cover frame 212, as shown in FIG. 2.

As indicated above, leaflets 211 are supported by frame 212. In this regard, leaflets 211 may be sewn to ribbon sections 214 using holes 216 formed along the length of ribbon sections 214. Alternatively, leaflets 211 may be attached to ribbon sections 214 by appropriate means, such as sutures, clips, staples or other fastening devices. Leaflets 211 may be made of suitable materials, including, but not limited to, bovine pericardium, equine pericardium, ovine pericardium, porcine aortic valve tissue, small intestinal submucosa (SIS), various biodegradable substrates for tissue engineered valves, and various relatively inert polymers, such as polyurethane.

In the illustrated embodiment of the present invention, each pair of engagement portion 30/coupling element 220 provides a protuberance-slot mechanism. However, it is also contemplated that the configuration may be reversed, wherein each coupling element 220 provides a protuberance and each engagement portion 30 provides a slot.

Fixed post 20 and floating posts 22 and 24 of docking station 10 are substantially the same, and therefore only floating post 22 will be described in detail with particular reference to FIGS. 1, 2, and 9. As discussed above, post 20 is comprised of mounting or engagement portion 30 and main body 40. Engagement portion 30 includes protuberance 32 in the form of a hook or tab. In the illustrated embodiment, a depression, recess or hole 36 is formed in engagement portion 30 to allow a tool (not shown) to engage and hold docking station 10.

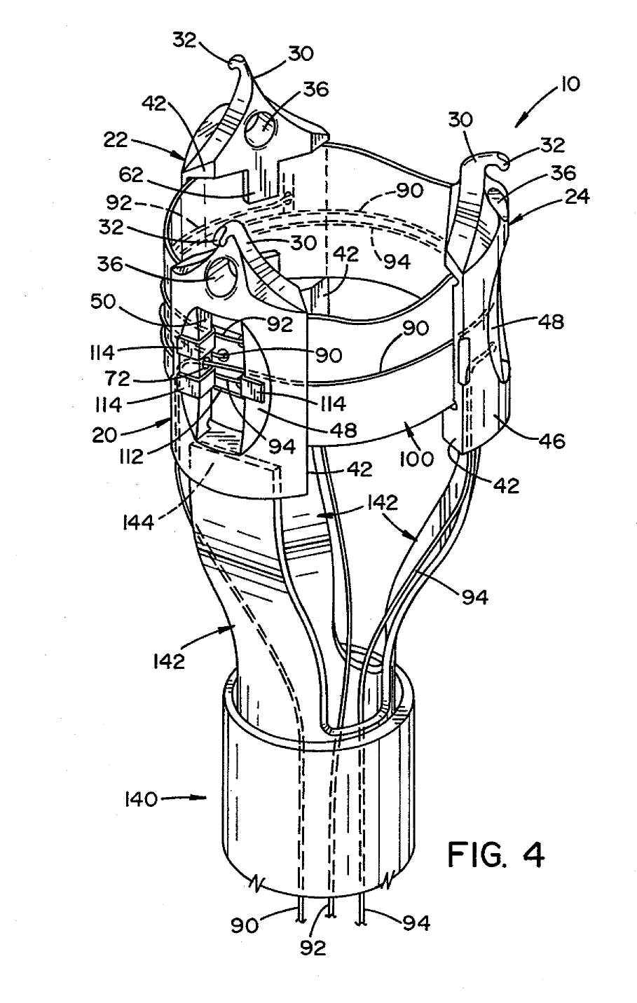

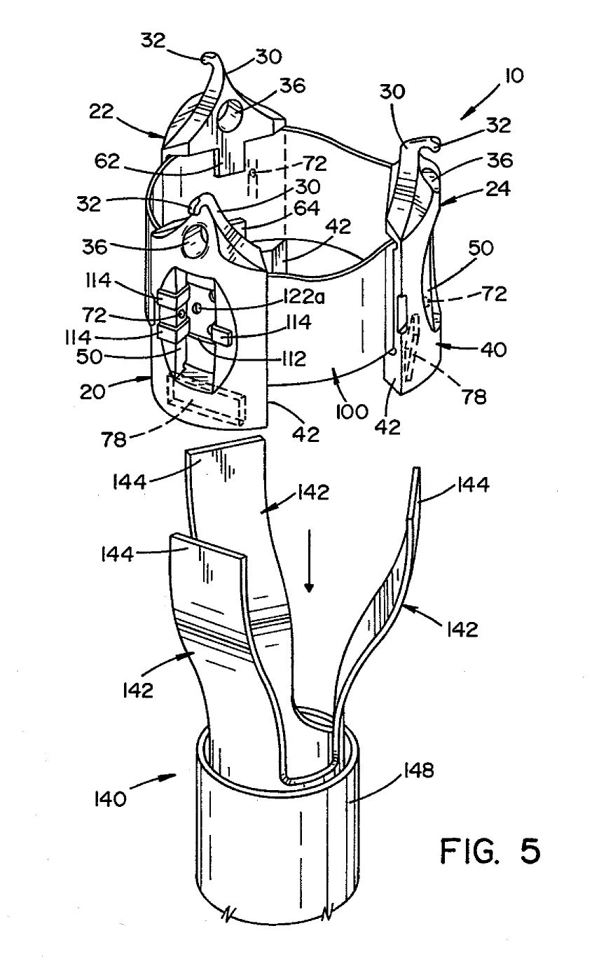

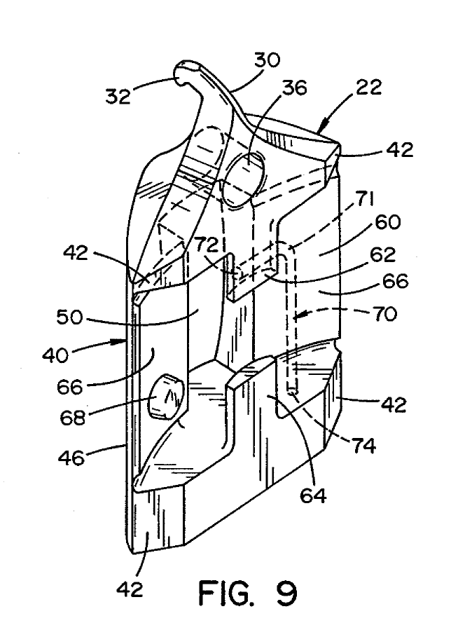

Main body 40 includes a pair of side or lateral surfaces 42, a curved face 46, and a central opening 50, as best seen in FIGS. 1 and 2. A pair of flat surface portions 48 are located adjacent to central opening 50. Main body 40 also includes a curved slot 60 that defines a curved face 66, a pair of opposing tabs 62, 64, an inward facing protrusion 68 that extends from face 66, and an internal channel 70, as best seen in FIG. 9. Slot 60 is dimensioned to receive band 100, and tabs 62, 64 capture band 100 within slot 60. In the illustrated embodiment, protrusion 68 takes the form of a pin. It should be noted that protrusion 68 of post 24 may be located at a different position than protrusion 68 of post 22. Furthermore, protrusion 68 is omitted from fixed post 20. Internal channel 70 has an inlet 72 and an outlet 74. In the illustrated embodiment, channel 70 has a turn or bend 71 that changes the direction of channel 70 from circumferential to axial. Accordingly, inlet 72 is at approximately a 90 degree angle to outlet 74. Channel 70 is dimensioned to receive a string as will be described below. Main body 40 may also include a plurality of engagement means for engaging with an installation tool 140 described below. In the illustrated embodiment, each engagement means takes the form of an axial recess 78, as best seen in FIGS. 4 and 5. It is contemplated that the engagement means may take alternative forms, e.g., a protuberance.

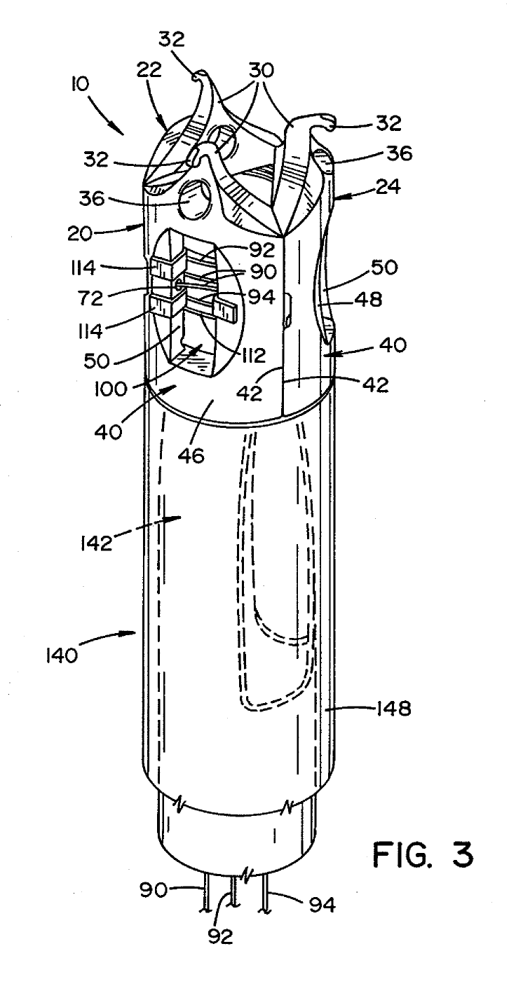

It is contemplated that installation tool 140 may take a wide variety of forms. A portion of installation tool 140 is shown in FIGS. 3-5 only for the purpose of illustrating operation of the present invention. As shown in FIGS. 3-5 installation tool 140 includes a plurality of moveable arms 142 and a sliding collar 148. Arms 142 are moveable between a collapsed position (FIG. 3) and an expanded position (FIGS. 4 and 5). Distal ends 144 of arms 142 are dimensioned to engage with recesses 78 of posts 20, 22, 24. In the illustrated embodiment distal ends 144 of arms 142 are dimensioned to be received into a recess formed in each post 20, 22, 24. Arms 142 move from the collapsed position to the expanded position by sliding collar 148 away from distal ends 144 of arms 142. It should be understood that installation tool 140 may take forms other than as shown herein, and is not intended to limit the scope of the present invention. For example, each arm 142 may be configured with a recess dimensioned to receive a protuberance (e.g., pin) extending from each post 20, 22 and 24.

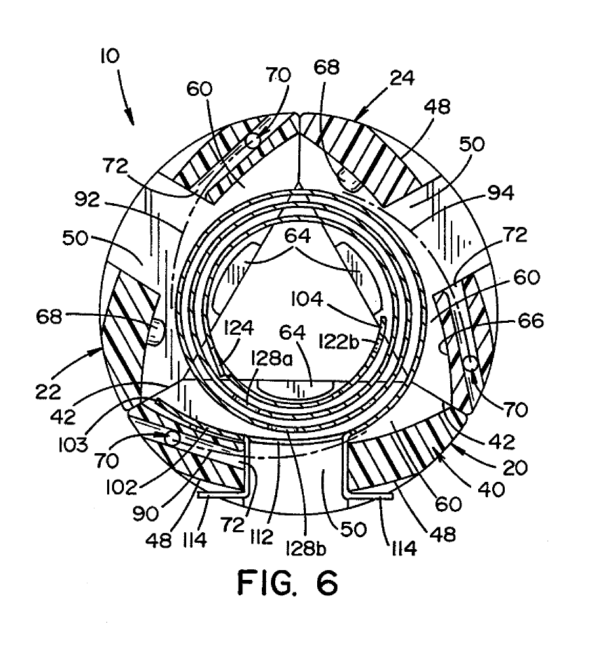

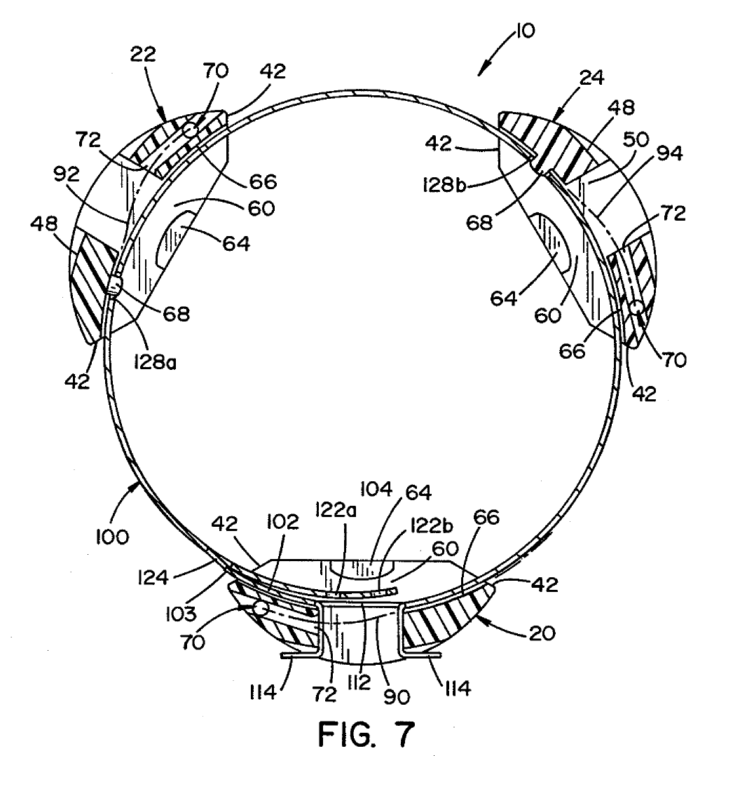

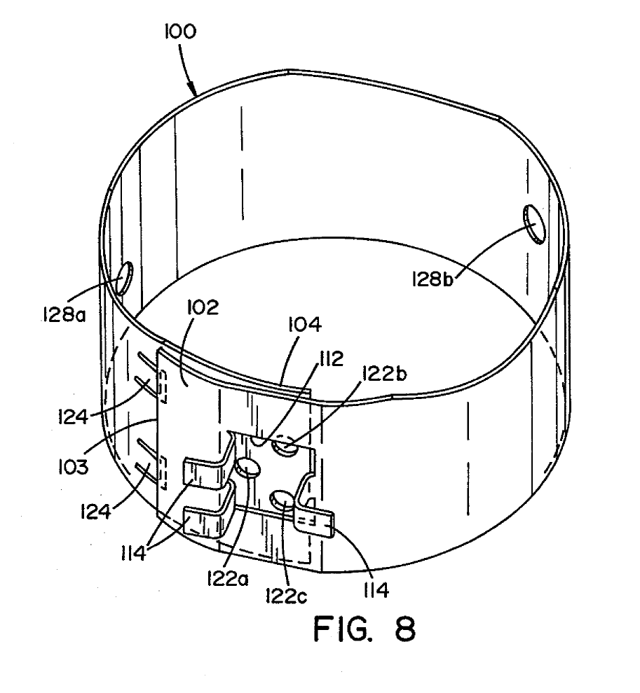

Band 100 will now be described with reference to FIGS. 6-8 and 10-11. Band 100 is a frame element that is moveable between a collapsed position and an expanded position. Accordingly, the diameter of band 100 is adjustable. In the collapsed position, band 100 is wound into a series of concentric circles to form a coil, as shown in FIG. 6. In a fully expanded position, band 100 is unwound to form a circular ring, as shown in FIGS. 7 and 8. It is contemplated that band 100 may be biased towards an expanded position, biased towards a collapsed position, or biased any amount within the range between the completely expanded and completely collapsed positions. In the illustrated embodiment, band 100 is biased towards the expanded position, thereby providing some elastic preload to the wound band 100.

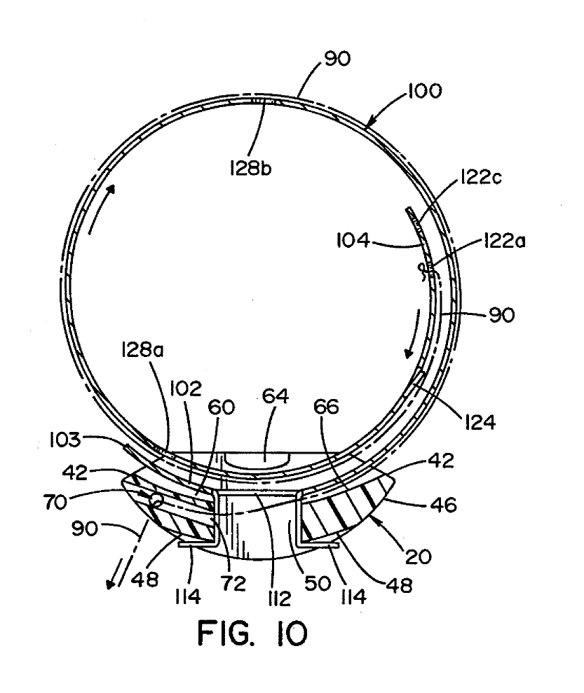

Band 100 has a first end 102 and a second end 104. As shown in FIG. 8, first end 102 is located on the outside of the circular ring formed by expanded band 100, while second end 104 is located on the inside of the circular ring formed by expanded band 100. An opening 112 is formed at first end 102. A plurality of L-shaped outwardly projecting tabs 114 are located adjacent to opening 112. Tabs 114 fix post 20 to first end 102 of band 100, as will be discussed below. A plurality of holes 122a, 122b and 122c are formed at second end 104 to fix respective wires or strings 90, 92 and 94 to band 100. FIG. 10 is simplified to show string 90 attached to band 100. FIG. 11 is simplified to show strings 92 and 94 attached to band 100. String 90 functions to unwind band 100 from a collapsed position to a fully expanded position. Strings 92 and 94 respectively function to properly locate posts 22 and 24 along the circumference of the ring formed by expanded band 100, as will be further explained below. One or more outward extending ears or tabs 124 are formed at second end 104 to lock band 100 in the fully expanded position, as best seen in FIGS. 6-8 and 10-11. In this regard, tabs 124 function as a locking member to engage or capture front edge 103 of band 100, thereby preventing band 100 from returning to the collapsed position after expansion. It should be appreciated that alternatives to tabs 124 may take the form of rivets. Holes 128a, 128b (FIG. 8) are formed in band 100 and are aligned and dimensioned to receive protrusions 68 of floating posts 22 and 24 (FIG. 7). As indicated above, protrusion 68 of floating post 22 and floating post 24 may be located at different positions of the respective floating post. As will be explained in further detail below, string 92 (associated with post 22) and string 94 (associated with post 24) are used to align protrusions 68 of posts 22 and 24 with respective holes 128a and 128b of band 100, thereby fixing posts 22 and 24 at a uniform distance from adjacent posts (e.g., separated at 120 degree increments) when band 100 is in the fully expanded position (FIG. 7). While band 100 has been described as a metal band (e.g., a spring steel ribbon), it is also contemplated that band 100 may be formed of an alternative rigid material such as a high strength plastic or other polymer material.

It is contemplated that all exposed surfaces of valve assembly 2 may be covered by a fabric cover (not shown) made of Dacron®, or other medical grade fabric, as in conventional bioprosthetic valves. The fabric cover facilitates installation of docking station 10 using a friction fit.

Assembly and operation of docking station 10 will now be described in detail. Posts 20, 22 and 24 are mounted onto band 100 by inserting band 100 through slot 60 of each post 20, 22 and 24. Unlike floating posts 22 and 24, post 20 is fixed to band 100. As best seen in FIGS. 6 and 7, L-shaped tabs 114 extend through central opening 50 of post 20 and capture main body 40 of post 20. In this regard, the distal ends of L-shaped tabs 114 are located adjacent to flat surface portions 48 of main body 40. L-shaped tabs 114 are dimensioned to prevent axial or lateral movement of post 20 relative to band 100.

As best seen in FIG. 10, a first end of string 90 extends through hole 122a to attach string 90 to second end 104 of band 100. Similarly, as shown in FIG. 11, a first end of string 92 extends through hole 122b to attach string 92 to second end 104 of band 100, and a first end of string 94 extends through hole 122c to attach string 94 to second end 104 of band 100. Strings 90, 92 and 94 are wound into a series of concentric circles along with band 100 as it is collapsed into a coil, as shown in FIG. 6. Accordingly, strings 90, 92 and 94 are wrapped between layers of band 100. The second end of string 90 extends through channel 70 of post 20. Similarly, the second end of strings 92 and 94 respectively extend through channel 70 of posts 22 and 24.

Band 100 is collapsed by winding band 100 into a coil, as shown in FIG. 6. When docking station 10 is in the fully collapsed position, lateral surfaces 42 of adjacent posts 20, 22 and 24 are in contact as shown in FIG. 3. Accordingly, posts 20, 22 and 24 form a compact tubular structure that surrounds collapsed band 100.

Installation tool 140 is used to locate and deploy docking station 10 at the surgical site where docking station 10 is to be installed, and facilitates movement of band 100 from the collapsed position to the expanded position. Installation tool 140 is engaged with docking station 10 by inserting distal ends 144 of arms 142 into axial recesses 78 of posts 20, 22 and 24. Collar 148 is initially located at distal ends 144 of arms 142, thereby maintaining arms 142 in the collapsed position. Accordingly, arms 142 keep band 100 in the collapsed position. Arms 142 of installation tool 140 are moved from the collapsed position to the expanded position by sliding collar 148 away from distal ends 144 (FIG. 4), thereby facilitating the unwinding and expansion of band 100 and the movement of docking station 10 from the collapsed position to the expanded position. Band 100 is shown in a partially expanded position in FIGS. 10 and 11 and is shown in a fully expanded position in FIGS. 4, 5 and 7.

In the illustrated embodiment, installation tool 140 facilitates expansion of band 100, while string 90 is used to further expand band 100 to the fully expanded position. Tension is applied to the second end of string 90 to pull string 90 out through outlet 74 of channel 70. As a result, band 100 is expanded to the fully expanded position wherein tabs 124 at second end 104 engage or catch front edge 103 of first end 102. As indicated above, tabs 124 prevent band 100 from returning to the collapsed position after expansion. After band 100 is fully expanded, strings 92 and 94 are manipulated in order to move posts 22 and 24 relative to band 100 such that respective protrusions 68 are aligned with holes 128a and 128b. Posts 22 and 24 are moved clockwise or counter-clockwise along band 100 by appropriate movement of strings 92 and 94. In the illustrated embodiment, posts 20, 22 and 24 are uniformly spaced apart along the circumference of the ring formed by expanded band 100.

Installation tool 140 is disengaged from docking station 10 by removing the distal ends 144 of arms 142 from axial recesses 78 of posts 20, 22 and 24 (FIG. 5). Strings 90, 92 and 94 are also removed from docking station 10.

Valve member 210 is coupled and uncoupled to/from docking station 10 through engagement and disengagement of coupling elements 220 and engagement portion 30. In the illustrated embodiment, lower slot 234 of each coupling element 220 is dimensioned to receive a respective protuberance 32 of each engagement portion 30, thereby coupling valve member 210 to docking station 10. As best seen in FIG. 2, protuberance 32 is captured between the lower surface of finger 228 of upper crossbar 226 and the upper surface of lower crossbar 224.

As discussed above, frame 212 is formed of an elastic material. Accordingly, frame 212 is dilated by outward deflection to disengage protuberance 32 of each engagement portion 30 from lower slot 234 of each coupling element 220. Consequently, valve member 210 is uncoupled from docking station 10. Coupling and uncoupling of valve member 210 to/from docking station 10 may be facilitated by use of a specialized tool set (not shown). Once valve member 210 is coupled to docking station 10, valve member 210 is secured such that it cannot unintentionally uncouple from docking station 10.

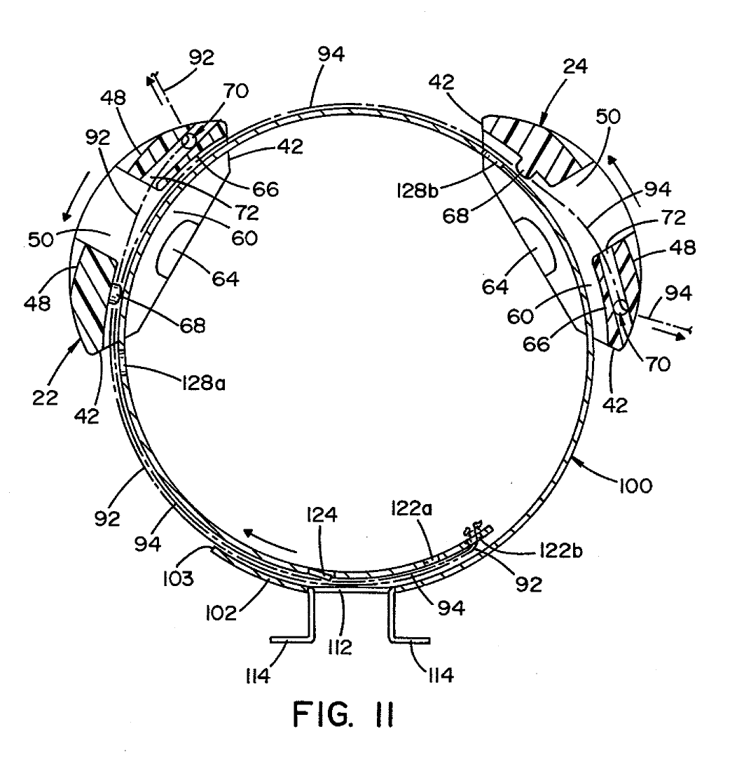

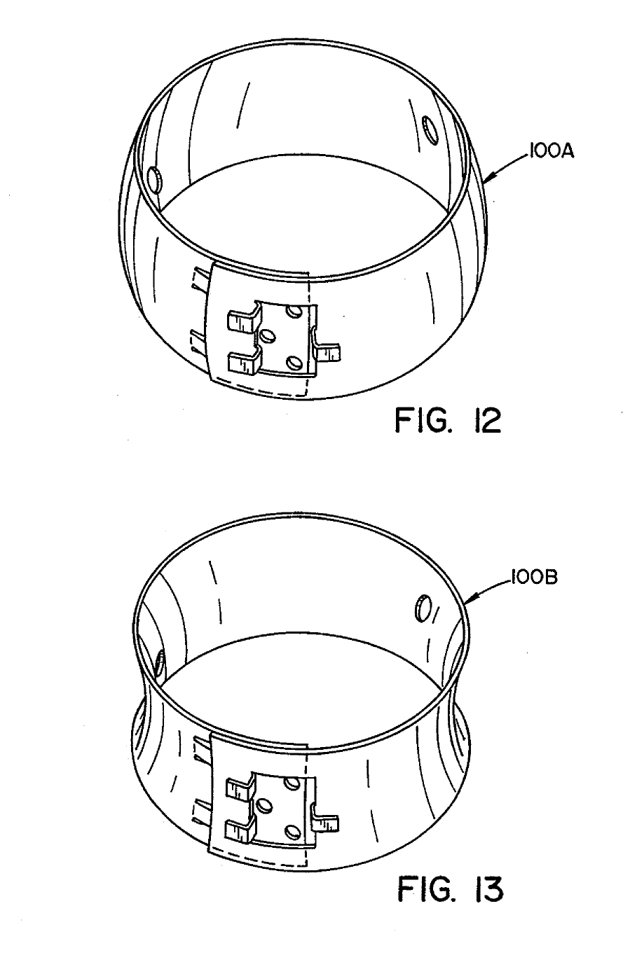

FIG. 12 illustrates a metal band 100A according to an alternative embodiment. Band 100A differs from band 100 in that it is convex shaped in the circumferential direction. In accordance with this embodiment, slot 60 of each post 20, 22 and 24 is dimensioned to receive convex shaped band 100A. FIG. 13 illustrates a metal band 100B according to still another alternative embodiment. Band 100B differs from band 100 in that it is concave shaped in the circumferential direction. In accordance with this embodiment, slot 60 of each post 20, 22 and 24 is dimensioned to receive concave shaped band 100B. The convex and concave shapes of bands 100A and 100B provide additional stiffness and rigidity.

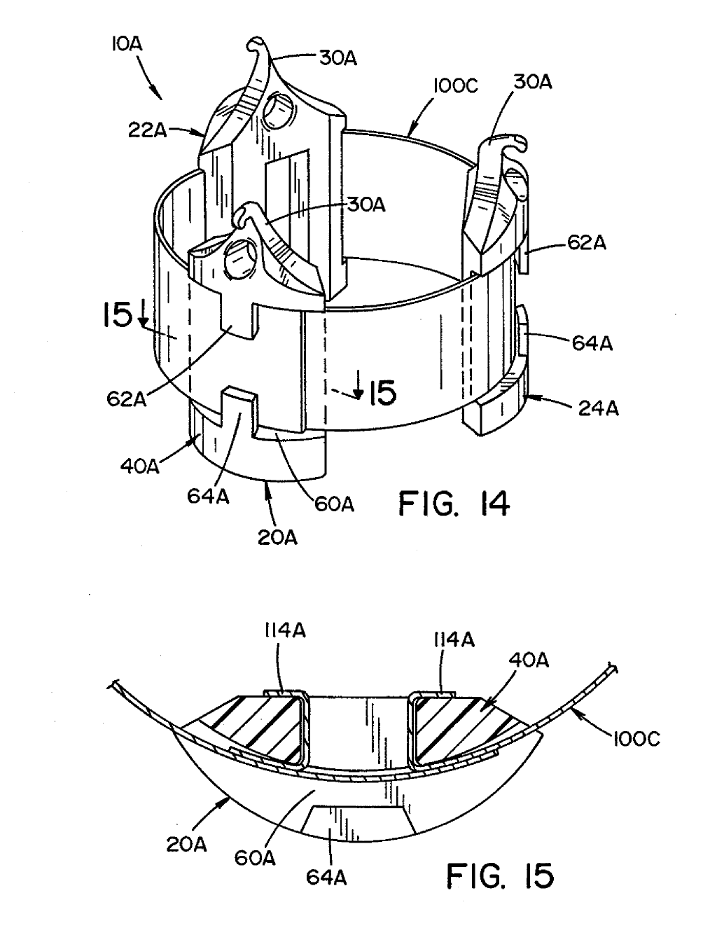

Referring now to FIGS. 14 and 15, there is shown a docking station 10A according to an alternative embodiment of the present invention. Docking station 10A generally operates the same as docking station 10. Docking station 10A is generally comprised of posts 20A, 22A and 24A and a metal band 100C. Posts 20A, 22A and 24A include an engagement portion 30A and a main body 40A. Engagement portion 30A is substantially the same as engagement portion 30 described above. Main body 40A is a generally reversed version of main body 40 described above. In this respect, main body 40A is configured such that curved slot 60A for receiving band 100C and opposing tabs 62A, 64A are located on the outer side of main body 40A, whereas curved slot 60 and opposing tabs 62, 64 of main body 40 are located on the inner side of main body 40.

Metal band 100C is substantially the same as metal band 100 described above. In this regard, band 100C is moveable between a collapsed position and an expanded position in a manner similar to band 100. Post 20A is fixed relative to band 100C by L-shaped tabs 114A. Posts 22A and 24A float relative to band 100C in the same manner as posts 22 and 24, as described above.

It should be noted that in order to improve clarity, some features of docking station 10A that are substantially the same as docking station 10 have been omitted from FIGS. 14 and 15.

According to the present invention, docking station 10 is adapted to be collapsible. As such, it can be inserted transapically during the initial surgery, using an appropriate tool set. Once collapsible docking station 10 is inserted and installed in a patient, valve member 210 can be coupled to docking station 10, also through apex, just like it is contemplated during a valve member exchange procedure.

An advantage of docking station 10 of the present invention, as compared to existing transapically insertable valves, is that valve member 210 will have a substantially circular, rigid configuration when coupled to docking station 10 (i.e., deployed). As discussed above, a non-rigid docking station 10 can give rise to the durability problems. Docking station 10 of the present invention can be inserted transapically and provides the required rigidity. Another important aspect of the present invention is that metal band 100 provides advantages over a wire cage In this regard, metal band 100 has greater radial stiffness, and thereby achieves circularity. Accordingly, the present invention provides a valve assembly that is durable, has a long usable lifespan, and allows exchange of a valve member.

The foregoing description is a specific embodiment of the present invention. It should be appreciated that this embodiment is described for purposes of illustration only, and that numerous alterations and modifications may be practiced by those skilled in the art without departing from the spirit and scope of the invention. For example, it is contemplated that the collapsible docking station and valve member may be formed as a single unit (i.e., one-piece rather than two-piece). Accordingly, in this alternative embodiment the present invention takes the form of a collapsible prosthetic valve having an adjustable-diameter frame and a leaflet set attached to the frame. The frame is wound into a coil to reduce the diameter of the valve during insertion and is unwound to increase the diameter of the valve during deployment. It is intended that all such modifications and alterations be included insofar as they come within the scope of the invention as disclosed or claimed or the equivalents thereof.

Having described the invention, the following is claimed:

1. A collapsible docking station engageable with a detachable valve member having valve leaflets, said collapsible docking station including:
a band moveable between a collapsed position and an expanded position; and
a plurality of posts adapted to engage with the detachable valve member.

2. A collapsible docking station according to claim 1, wherein said band forms a coil comprised of a plurality of concentric circles in the collapsed position and forms a circular ring in the expanded position.

3. A collapsible docking station according to claim 1, wherein said plurality of posts includes at least one post fixed to the band.

4. A collapsible docking station according to claim 3, wherein said at least one post fixed to the band includes a channel for receiving a string attached at one end to said band for expansion of said band.

5. A collapsible docking station according to claim 1, wherein said plurality of posts includes at least one post fixed to the band and a plurality of floating posts moveable relative to the band.

6. A collapsible docking station according to claim 5, wherein said collapsible docking station includes a plurality of strings attached to the band to respectively adjust the position of the plurality of floating posts.

7. A collapsible docking station according to claim 6, wherein each of said plurality of floating posts includes a channel for receiving a respective string.

8. A collapsible docking station according to claim 1, wherein said collapsible docking station includes at least one string attached to the band to expand said band to the fully expanded position.

9. A collapsible docking station according to claim 1, wherein said band includes a locking member for locking said band in a fully expanded position.

10. A collapsible docking station according to claim 1, wherein said band has a concave or convex shape in the circumferential direction.

11. A collapsible docking station according to claim 1, wherein said band includes engagement means for engaging with a tool for facilitating movement between the collapsed position and the expanded position.

12. A collapsible docking station according to claim 1, wherein each of said posts include an engagement portion for coupling the detachable valve member to said collapsible docking station.
